(19) United States
(12) Patent Application Publication
(10) Pub. No.: US 2025/0114066 A1
(43) Pub. Date: Apr. 10, 2025

(54) DEVICE AND SYSTEM FOR EXCHANGING ENERGY AND/OR DATA, IN PARTICULAR FOR LOCALISATION PURPOSES, WITH AT LEAST ONE IMPLANT AND/OR AT LEAST ONE ORGAN IN A HUMAN OR ANIMAL BODY AND/OR AN APPARATUS EXTERNAL TO THE BODY VIA PIEZOELECTRIC AND/OR CAPACITIVE TRANSDUCERS

(71) Applicant: Caranx Medical SAS, Paris (FR)

(72) Inventors: Marc LACROIX, Louvain-La-Neuve (BE); Patrick COULOMBIER, Paris (FR); Philippe POULETTY, Paris (FR); Constantin KREMPP, Clamart (FR); Claire COROT, Lyon (FR)

(73) Assignee: Caranx Medical SAS, Paris (FR)

(21) Appl. No.: 18/294,843

(22) PCT Filed: Aug. 3, 2022
(86) PCT No.: PCT/EP2022/071792

§ 371 Date: Feb. 2, 2024

(30) Foreign Application Priority Data
Aug. 4, 2021 (FR) 2108490

(51) Int. Cl. A61B 8/08; A61B 8/00; A61B 34/20; A61B 90/00; B06B 1/06

(52) U.S. Cl. CPC A61B 8/0841; A61B 8/4488; A61B 8/4494; A61B 34/20; B06B 1/0622; A61B 2034/2063; A61B 2090/3925

(57) ABSTRACT

Device and system for exchanging energy and/or data, in particular for localization purposes, with at least one implant and/or at least one organ in a human or animal body and/or a device external to the body, via piezoelectric and/or capacitive transducers.

Device (1) for exchanging energy and/or data, in particular for localization and optionally tracking purposes, with at least one implant in or on a human or animal body and/or with at least one organ of a human or animal body and/or a device external to a body, comprising:
a substrate (10);
at least one group of a plurality of piezoelectric (3) and/or capacitive transducers, supported by the substrate, each transducer forming an elementary piezoelectric or capacitive transceiver configured to emit ultrasound signals and/or receive other ultrasound signals;
at least one electronic switching circuit (4), supported by the substrate or by a support, whose inputs are designed to be supplied with ultrasound signals, whose outputs are each connected to one of the transducers, and whose control bus(es) (21) are designed to be connected to at least one command processor.

### TECHNICAL AREA

The present invention relates to the field of medical diagnosis and, where appropriate, therapy using ultrasound waves.

More specifically, it concerns the location and, where appropriate, tracking of an organ or implant in a human or animal body, whether the implant is passive, semi-active or active.

For the purposes of this invention, “implant” is taken to mean an object/device introduced at least temporarily under or on the skin, in a tissue, in an organ, in a vein, artery, etc. of a human or animal body, for diagnostic, surgical or medical purposes, or for therapeutic purposes.

For the purposes of this invention, “passive implant” is taken to mean an implant that does not participate in its localization, except through the echo or reverberation it generates from ultrasound emitted from outside the human or animal body into which it is introduced.

By “semi-active implant”, we mean here and in the context of the invention, an implant which is capable of functioning as a transceiver adapted to receive ultrasound emitted from outside the human or animal body into which it is introduced, and/or of emitting other signals autonomously or in response to the signals received.

By “active implant”, we mean here and in the context of the invention, an implant which is capable of functioning as a transceiver adapted to receive ultrasound emitted from outside the human or animal body into which it is introduced, of processing it, of emitting other signals potentially including other information gathered locally, i.e. in the area of the human or animal body surrounding it and/or of executing actions locally, such as electrical, mechanical or chemical activation, displacement, change of shape.

In the context of the invention, an implant can be embedded, i.e. be an autonomous electronic and computer system, capable of movement within a human or animal body, and capable of detaching from its initial position either programmed or by chance.

### Previous Technique

Medical imaging is a diagnostic and screening tool, as well as a preparation for surgery or a tool for monitoring the evolution of a pathology.

Here, we briefly review the main principles of the various existing non-invasive medical imaging techniques:
Radiography uses X-rays that pass through the human body, being more or less absorbed by body tissues, depending on their density. A photographic film or a more sensitive electronic detector reveals or digitizes the image;
MRI (an acronym for Magnetic Resonance Imaging) uses the property that the nuclei of certain atoms, notably those of a human or animal body, have of emitting signals when subjected to a magnetic field and a radiofrequency pulse in a given range. The resulting images are cross-sections of the area(s) subjected to resonance;
Ultrasound involves placing an ultrasound transceiver probe on a human or animal body, with the ultrasound being sent into the body's tissues, and the recorded echoes being signatures of the obstacles they have encountered. There are several types of probe, operating at different frequencies depending on which part of the body's anatomy is to be explored.

Typically, the frequencies used in ultrasound are:
from 1.5 to 4.5 MHz in common use for deep exploration, as for the abdomen and pelvis, with a definition of the order of a few millimeters;
of the order of 5 MHz for intermediate anatomical parts, such as a child's heart, with sub-millimeter resolution;
of the order of 7 MHz for the exploration of small parts underlying the skin, such as arteries or veins, with a resolution close to a tenth of a millimetre;
from 10 to 18 MHz for the study of small animals in research, but also, in the medical field, for superficial imaging of parts close to the skin;
up to 50 MHz for eye biomicroscopy equipment.

Another existing technique, positron emission tomography (PET), is important in dynamic diagnostics, as it enables us to visualize not the shape but the function of an organ over time, just like “functional” MRI.

In the field of medical diagnostics and, where appropriate, therapy, new clinical needs have been identified:
localization of at least part of medical devices during interventional radiology procedures; during such procedures, the devices used to treat the patient are inserted in the least invasive way possible, with very low visibility inside the body;
monitoring the spatial evolution of the positioning and/or dimensions of an implant, in real time, in an ambulatory setting, typically on time scales of several days or several months, or shorter, such as monitoring an interventional procedure in real time, without requiring the assistance of qualified personnel. For example, this may involve monitoring the spatial evolution of an implant to check for proper attachment or unwanted migration, or monitoring the degradation of an implant;
surgical image registration, monitoring the position of objects or organs during surgery, such as interventional cardiology. More specifically, during certain surgical procedures, it is necessary to know the location of organs, by means of fiduciary markers, or medical devices adapted for interventions when the vision of the practitioner(s) on the operating field is impossible or degraded. Knowing the location of the organs would enable them to be aligned with the patient's preoperative or intraoperative images, in order to:
monitoring the positioning of a device for delivering molecules and/or active ingredients, or of a therapy device in precise anatomical zones of a human or animal body;
local stimulation of a tissue or organ;
real-time assistance from autonomous mobile implants to help navigate through the vascular network of a human or animal body.

However, currently known devices/equipment implementing the above-mentioned medical imaging principles do not meet these needs.

More generally, these existing imaging devices/equipment do not enable objects introduced into a human or animal body to be tracked in real time on timescales of the order of a month, and also autonomously, whatever the principle used among X-rays, MRI or ultrasound.

Moreover, they require the intervention of qualified personnel to interpret the information received.

Finally, each of these devices/equipment/devices has intrinsic limitations that can be listed as follows:
X-ray radiation risks for patients and operating personnel;
safety limits relating to ionizing radiation or strong magnetic fields, making it impossible to use in the presence of an interventionist or instruments at the patient's bedside;
possible localization, generally in two dimensions (2D), and therefore the impossibility of obtaining information on the depth of an object relative to the body into which it is introduced (X-rays, ultrasound in interventional situations), even though X-rays can provide organ sections and 3D visualization in complete but static scans;
poor temporal resolution: in some cases, contrast agents such as iodine do not provide a clear view of anatomical structures at all times (X-rays);
low spatial resolution, i.e. the difficulty or impossibility of distinguishing anatomical structures that are too small, e.g. less than a few millimetres (X-rays, MRI);
complexity and high cost (MRI, X-rays);
the need to systematically use a gel between the ultrasound probe and the body of the patient to be imaged.

The lack of up-to-date equipment to meet the new needs identified can be explained by the inherent constraints of the human and animal body.

By its very nature, the human body is made up of various organs and tissues with different characteristics (muscle, bone, skin, blood, etc.). In particular, the speed of ultrasound propagation varies according to the materials, and therefore the tissues, encountered during an ultrasound scan. When a wave passes from one medium to another, part of it is reflected. As a result, a wave propagating through the human body is reflected multiple times, complicating the processing of the signal containing the localization information. In other words, at each distinct tissue/organ interface, part of the wave is reflected. Ultrasound wave disturbance results in poorer ultrasound image quality.

What's more, to locate an object in the body, you need to have a description (cartography) of the body and be able to place the object on it. In the case of the human body, mapping is based on imaging, carried out at a point in time before a patient's treatment. However, the map can be modified in real time according to body movements/deformations (articulated limbs, deforming soft tissues . . . ). Thus, locating an object and registering it in relation to a moving human body is not an easy task.

Similarly, if you want to operate a real-time localization system over several days, it needs to be linked to the human or animal body. At least one part of the system must therefore conform as closely as possible to the patient's deforming body.

The tissues of a human or animal body must be subjected to physical phenomena, as well as to non-toxic/non-destructive powers and/or frequencies.

Several developments are currently underway in transcranial ultrasound imaging, as an alternative to MRI or X-ray CT for diagnosing cerebral pathologies such as stroke. More generally, transcranial ultrasound with ultrasound stimulation is relevant to neuroscience and neurosurgery as a therapeutic modality for many neurological and psychiatric conditions, including epilepsy, depression, anxiety disorders, movement disorders and traumatic brain injury.

For example, U.S. Pat. No. 6,239,724B1 discloses a device and method for positioning a medical instrument and/or directing a medical procedure in a patient's body using ultrasound signals. The disadvantages of the disclosed device are numerous, including the need for an active implant that must also be fixed, which severely restricts its field of application.

Patent application US2019/0308036A1 discloses a conformable and portable ultrasound transducer patch for the delivery, monitoring and spatio-temporal control of localized focused transcranial ultrasound, the patch comprising a continuous layer of piezoelectric material overlying a continuous layer, The patch comprises a continuous layer of piezoelectric material overlying a continuous layer, which defines the geometric resolution of the various transducers by means of conductive vias which open out onto the latter, each of which is connected by one of the vias to an electronic switch which sends back to a receiving circuit or a transmitting circuit.

Patent U.S. Pat. No. 10,231,712B1 discloses a method and related system for ultrasound therapy, in which ultrasound signals are focused by a concave-shaped transmitter and the received signals are processed using an image reconstruction method.

Patent applications US2018/37653 and WO2020/176830 relate to improvements in ultrasound investigation techniques for exclusively cardiological applications, involving a wearable, flexible patch whose geometric configuration is determined to correct received signals or images, so as to obtain sharp images in the end.

The processes and systems disclosed in these last two documents are far from being completely satisfactory in relation to the new needs identified.

In particular, they do not provide sufficiently high accuracy for implant localization in many applications, and there is no guarantee of being able to track in real time the positioning of an implant that might move and/or change.

More generally, the processes and systems do not enable energy and/or data exchange with an implant or organ of a human or animal body, or even with a device external to the body, which is sufficiently precise and at a sufficiently high level in the case of energy exchange.

The aim of the invention is therefore to meet the new clinical needs identified by overcoming the drawbacks/limitations mentioned above.

### Explanation of the Invention

Thus, the present invention relates, according to a first of its aspects, to a device for exchanging energy and/or data, in particular for localization and, where appropriate, tracking purposes, with at least one implant in or on a human or animal body and/or with at least one organ of a human or animal body, such as part of a nerve, and/or with at least one external device to a human or animal body comprising:
a substrate, preferably applied to the body from the outside;
at least one group of a plurality of piezoelectric and/or capacitive transducers supported by the substrate, each transducer forming an elementary piezoelectric or capacitive transmitter/receiver configured to emit ultrasound signals from the outside and/or inside of the body to the inside and/or to the outer surface of the body and/or to the external device and/or to receive other ultrasound signals including those from the inside of the body and/or those from the external device;
at least one electronic switching circuit, of the matrix switch type, supported by the substrate, or by a support fixed to the substrate, the inputs of which are intended to be supplied with ultrasound signals, possibly of different phases, the outputs of which are each connected to one of the piezoelectric and/or capacitive transducers, and the control bus(es) of which are intended to be connected to at least one command processor;
device in which the piezoelectric and/or capacitive transducers are configured to operate as a phased array ultrasound antenna.

Unlike prior art probe ultrasound devices and systems, a user of a device according to the invention has no manual or automated scanning of the body surface to perform, as the piezoelectric transducers of a group can operate as a phased array ultrasound antenna directly fed by a matrix switch integrated or fixed on the same support (substrate), the scanning of the body surface taking place in particular by the operation of the matrix switch.

One mode of operation of the invention is to identify the position of piezoelectric and/or capacitive transducers relative to references or targets internal to the human or animal body. Thanks to the invention, any variation in the position of a reference or target that is identified by the distances directly, also includes variations in the various layers located between the transducers and the reference/target chosen to calibrate the device.

This is fundamentally different from devices in the prior art, such as those in US2018/37653 and WO2020/176830, which operate by image adaptation or calculations based on information between transducers until a sharp image is obtained.

In other words, the device can be used to obtain the precise position of an object or target, implant, etc., i.e. the actual coordinates required for absolute or relative positioning, without the need for image processing (adaptation, calculation).

In practice, this guarantees fast, reliable detection of an object (implant) or organ within the complex environment of a human or animal body, which may, for example, contract a muscle, making it thicker, and/or lengthen, mobilizing certain organs or skeletal landmarks, etc.,

For the purposes of this invention, “command processor” refers to any electronic device capable of executing a program to apply commands to another device.

For the purposes of this invention, “transducer” or “transmitter/receiver” refers to any device capable of transmitting or receiving one or more ultrasonic signals, or of performing both transmission and reception functions.

By “frequency”, “ultrasound signal” or “ultrasonic wave”, we mean here and in the context of the invention, any pressure waveform (acoustic waves) whose frequency lies beyond the acoustic range, i.e. most often from 20 kHz to 50 MHz, including modulated or complex waves, multi-frequency waves, wavelets and more generally any waveform that can be synthesized by analog and/or digital means. Where reference is made to a frequency f, this refers to a characteristic frequency selected from the wave spectrum.

Thus, the invention essentially consists of a device whose substrate supports a multitude of piezoelectric and/or capacitive transducers which emit and/or receive ultrasound which can be used to exchange data, in particular for fixed or tracked localization, and/or energy with a target, constituted by an implant, an organ of a human or animal body, such as a part of a nerve or a bundle of nerve fibers, or an external device in the vicinity of a human or animal body. By extension, the target can be an anatomical position relative to a fixed body landmark, such as a bone, or a fiduciary landmark. The piezoelectric and/or capacitive transducers function as a phased array ultrasound antenna, with the surface of the human or animal body being scanned in particular by the use of the matrix switch.

If the device is to be used for exchanging data and/or electrical energy with an implant or organ, it can be pre-detected with a portable device and then, once an area of the body has been detected where the ultrasound signals are optimal, the device substrate can be adjusted, in particular by means of a patch.

Ultrasound emission from transducers can be unidirectional, with three types of signal as follows:
information signals (data communication) which are transmitted to the implant or to any external device in the vicinity and within the reception field of the emitted ultrasound;
signals for transferring electrical energy (power) to an implant or to any external device within range of the transducers, in particular by high-power or low-power emission, by a concentrated beam or not
modulated signals, i.e. signals whose amplitude and/or frequency is modified, in order to generate local treatment and/or diagnostic/therapeutic/medical conditions, such as local conditions of heat, pressure, electrochemistry . . . ;

Data exchange and energy transfer signals can be generated separately or, more generally, simultaneously. For example, a continuous ultrasonic wave, modulated in frequency, phase, amplitude, or pulsed, is a signal that can be converted back into energy and decoded to extract the data signal.

Ultrasound emission from the transducers can also be bidirectional, with data and/or energy reception signals emitted by an external device within the transducers' reception range, in addition to the above three types of signal.

The energy received can be managed, i.e. received, stored and used locally in any implant or external device, or the patch itself, which can be energy self-sufficient.

Like a single device, a multi-device system can transmit unidirectionally or bidirectionally with the signal types mentioned above.

More specifically, communication can be bidirectional in “network” mode or in “low-power network” mode: each device can then be a relay, for example in the sense of the BLE (Bluetooth Low Energy) standard, or in the sense of the IoT (the Internet of Things), and all the transducers of all the devices can optionally act as an independent energy source, i.e. transmit a beam of ultrasonic waves which is intended to be received and used as an energy source in the receiver.

Intermediate equipment can be used as a relay between each device and the implant or device external to the body to relay the data/energy sent. With at least one device according to the invention and possibly one or more implants, all types of biomarkers from a human or animal body can be monitored, such as:
physiological (eating, sleeping, . . . );
biochemical (chemical concentration . . . );
electrical (electrical recordings, impedance measurements, etc.);
mechanics (rigidities, deformations . . . );
physics directly related to the propagation of an ultrasonic wave in the body, such as celerity, spectrum, power, refraction, dispersion, reflection, transmission . . .

Various monitoring paradigms are possible, such as the active analysis of body tissues or the passive reception of signals from them, by means of monomodal or multimodal information.

Biomarker signal capture, conditioning and recording can be carried out by an implant according to the invention and communicated to the device in synchronous mode, i.e. in real time, or asynchronously, for example once a day, asynchronous mode being initiated at the request of the device or on the basis of a specific signal, coming from the implant or the device or by an HMI (acronym for Human Machine Interface) controlled by an operator, the patient, . . . .

Preferably, most recordings are made by the device to reduce the complexity of an implant in accordance with the invention.

Even more preferably, most of the data processing and memory storage components are in the device, to reduce the complexity of an implant conforming to the invention.

### Device

### Substrate

As mentioned above, a device according to the invention comprises a substrate to be applied preferably against the body from the outside.

The substrate itself can be applied to any tissue on a human or animal body, directly or indirectly in contact with the skin, on mucous membranes or tissues, subcutaneously . . .

The substrate can also be applied to elements outside the human or animal body for which we wish to locate an organ and/or into which we wish to introduce an implant with which we wish to exchange data, in particular in order to locate it, and/or energy. For example, the substrate can be applied to an intervention and/or manipulation robot, to a structural part of an operating room, to other people such as nursing staff . . .

The substrate can be made of any material suitable for the applications covered by this application, in particular a material compatible for contact with the human or animal body, in particular human skin. By “material compatible for contact with the body”, we mean here and in the context of the invention, any material having the ability not to interfere, i.e. being chemically and electrically inert with respect to the surface of the human or animal body. Advantageously, the substrate can be made of biocompatible material, e.g. in accordance with ISO 10-993.

The substrate can be flexible, semi-rigid or rigid. It may be in the form of a cream or gel.

According to a preferred embodiment, the substrate is made of soft, flexible and/or stretchable material(s), which advantageously enables it to conform to the part of said human or animal body against which it is applied.

In a preferred embodiment, the substrate consists of one or more layers of flexible and/or stretchable film material.

For the purposes of the present invention, a “soft” or “flexible” material is defined as a material with a good capacity to resist stress, which deforms easily and can return to its original shape after being deformed. For the purposes of the present invention, the term “stretchable” material is used more particularly to designate a material capable of undergoing significant elongation, in particular beyond 60% of its initial length, while recovering its initial length after elongation.

The softness or flexibility of a material can be characterized by its Young's modulus, or longitudinal modulus of elasticity.

Thus, a material suitable for implementing the invention is more particularly a material with a Young's modulus less than or equal to 5.0 GPa, in particular ranging from 0.05 to 4.0 GPa, more particularly ranging from 0.15 to 3.5 GPa, for example from 0.3 to 3 GPa. Furthermore, the thickness of the material film can also have an impact on its flexibility. Thus, at constant Young's modulus, the thinner the material film, the softer or more flexible it will be.

This is why the thickness of the film(s) of material(s) forming the substrate is preferably less than or equal to 300 μm, in particular between 25 μm and 300 μm, more particularly between 50 and 200 μm.

Thus, the substrate is advantageously made up of one or more layers of a film of flexible and/or stretchable polymeric material chosen from polyethylene terephthalate (PET), polyethylene naphthalate (PEN), polyvinyl chloride (PVC), polyvinylidene chloride (pvdc), poly(p-xylylene) (parylene), silicone, polysulfone, fluoropolymers such as polytetrafluoroethylene (PTFE), ethylene-chlorotrifluoroethylene (ECTFE) copolymers, ethylene-tetrafluoroethylene (ETFE) or ethylene-perfluoropropylene (FEP) copolymers, or polychlorotrifluoroethylene (PTFCE), a polyimide, a polyurethane (PU), a polycarbonate (PC), a polyester, a polyetherketone (PEEK), polymethylpentene (PMP), polyphenylene sulfide (PPS), polyamide, such as polyamide 6-6 (nylon), polyamide-imide (PAI), polylactic acid (PLA), and copolymers of at least two of the polymers defined above.

The polymer(s) used to produce the substrate can be chosen from thermoplastic or thermoset polymers, such as bakelite, epoxy or polyurethane.

The advantage of choosing a thermoset polymer is that it can be molded cold or at low temperature, usually below 80° C.

The polymer film may optionally be filled, i.e. it comprises at least one filler dispersed in the polymer film, e.g. an inorganic filler, in particular selected from silica, aluminum hydroxide, aluminum oxide, magnesium oxide, barium sulfate, magnesium, calcium, barium or strontium carbonate, clays, carbon black, titanium dioxide, and mixtures thereof. Other examples of fillers suitable for polymer films include fibers, whether woven or not.

It is within the skill of the person skilled in the art to adjust the filler ratio in the polymer material, in particular according to the size and nature of these fillers, in order to obtain a substrate with the desired mass and rigidity.

When the filler(s) are present, they can be implemented so as to obtain a filler content in the material ranging from 1% to 70% by weight, in particular from 5 to 50% by weight, relative to the total mass of flexible and/or stretchable polymer material.

The polymer film can also include any additive customary in the field of polymer materials, in particular chosen from plasticizers, stabilizers, colorants, etc.

The substrate can consist of one or more layers of a film of flexible and/or stretchable polymeric material as defined above, coated with a film of rigid material whose thickness is sufficiently thin for the coating film itself to be flexible and/or stretchable. In particular, the coating film can have a thickness of 400 μm or less, especially between 0.001 and 200 μm.

The coating film can consist of a sheet of metal, glass, especially chemically tempered glass, or mineral, such as a thin layer of tin-doped indium oxide (ITO) deposited by CVD or PVD (Chemical/Physical Vapor Deposition).

In another embodiment, the substrate can be rigid or semirigid.

According to this embodiment, the device comprises an interface layer adapted to conform to the portion of said body against which the substrate is applied.

Piezoelectric and/or Capacitive Transducers

As mentioned above, a device according to the invention comprises at least one group of a plurality of piezoelectric and/or capacitive transducers supported by the substrate.

### Group(s)

Preferably, the unit is square, rectangular, rhombus, hexagonal or octagonal when viewed from the front. It goes without saying that the general shape can be bevelled or rounded at at least one of its corners.

### Transducers

Each transducer forms an elementary piezoelectric or capacitive transmitter/receiver and is configured to transmit ultrasound signals from the outside and/or inside of the body to the inside and/or outside surface of the body, and/or to receive other ultrasound signals including those from the inside of the body.

Ultrasound signals can be received from inside the body to locate the implant. They may also be signals from elements outside the human or animal body housing the implant. For example, signals from one or more other devices according to the invention to enable direct communication between them. They may also be signals from separate elements of a device according to the invention and from the body housing the implant. For example, signals can be received from a robot or a remote monitoring structure, to enable synchronized geolocation between a patient whose body incorporates an implant located by a device according to the invention and the outside world.

Ultrasound signals can be exchanged with a fixed or mobile element in a diagnostic and/or surgical room, on the patient or on a robot, or between a medical practitioner and the patient, or more generally with any element in the environment external to the device.

In general terms, too, any ultrasound can interact with the transducers of a device according to the invention, including external signals that could serve as direct commands.

The piezoelectric and/or capacitive transducers in the group are preferably identical, evenly distributed over the substrate surface.

Each piezoelectric or capacitive transducer can be square, rectangular, triangular, hexagonal or circular in cross-section. The thickness of a block is adapted to the frequency, the target distance from the implant and the intensity of the ultrasound signals you wish to/can emit.

Advantageously, each piezoelectric or capacitive transducer has surface dimensions ranging from 10×10 μm to 1000×1000 μm, preferably ranging from 100×100 μm to 300×300 μm, most preferably equal to 200×200 μm.

Advantageously, the spacing between two adjacent piezoelectric transducers is less than 1 mm, preferably less than 100 μm, most preferably less than 50 μm.

The group can comprise a number of N, for example a multiple of a number of 64, without this number N having to be a power of 2, piezoelectric transducers distributed over a square or rectangle, preferably a number of 1024 piezoelectric transducers distributed over a square of 8×8 mm.

Each transducer is preferably configured to operate at a resonant frequency of between 1 and 50 MHz, preferably between 5 and 25 MHZ.

### Elementary Transducer Material

Each elementary transducer can be made of any material suitable for forming a piezoelectric or capacitive transmitter/receiver, which can be configured to transmit ultrasound signals from the outside and/or inside of the body to the inside and/or outside surface of the body and/or receive ultrasound signals from the inside of the body.

Such piezoelectric and/or dielectric materials are known to those skilled in the art, and can be chosen from ceramics, polymers or composite piezoelectric materials.

The nature of the piezoelectric or dielectric material is not limited, as long as it has an acoustic impedance, a dielectric constant, a coupling constant or a piezoelectric constant suitable for the realization of the invention.

Thus, according to one embodiment, the piezoelectric material of the transducers is a ceramic.

Ceramics suitable for the invention can be selected from lead titanate zirconate (PZT), in particular PZT-4 or PZT-5H, modified lead titanate (Pb, Ca)TiO₃, (Pb, Sm)TiO₃, (Ba, Sr)TiO₃ (BST), barium titanate BaTiO₃ or potassium sodium niobate (KNN).

In a preferred embodiment, the piezoelectric material of the transducers is a polymer.

Preferably, a polymer suitable for the invention comprises at least one constituent unit derived from vinylidene difluoride (PVDF).

Preferably, the polymer suitable for the piezoelectric transducer comprises at least one unit derived from vinylidene difluoride and at least one unit of formula (I), distinct from the unit derived from vinylidene difluoride:

—CR¹R²—CR³R⁴— (I)
formula (I) in which:
R¹ and R² independently of one another represent H, F or an optionally partially or fully fluorinated (C₁-C₃)alkyl group;
R³ represents F, Cl, Br, I, —C(O)OH, —CH₂—C(O)OH, —C(O)O—CH₃, —S(O)OX, Si(OR)₃, phosphonate or an optionally partially or fully fluorinated (C₁-C₃)alkyl group;
R⁴ represents F, Cl, Br, I, —C(O)OH, —CH₂—C(O)OH, —C(O)O—CH₃, —S(O)OX, Si(OR)₃, phosphonate, an optionally partially or fully fluorinated (C₁-C₃)alkyl group, or a photoactive group of the formula —Y—Ar—R⁵,
X representsF, —OK, —ONa, —OH or —Si(OR)₃,
R represents a methyl, ethyl or isopropyl group,
Y represents an atom selected from O and S, or an —NH— group,
Ar represents a (C₅-C₁₁) aryl group, and
R⁵ represents a monodentate or bidentate group containing from 1 to 30 carbon atoms.

Preferably, the polymer can be of formula (I) in which:
R¹ and R² independently represent H or F;
R³ represents F, —C(O)OH, —CH₂—C(O)OH, —C(O)O—CH₃ or an optionally partially or fully fluorinated (C₁-C₃)alkyl group;
R⁴ represents F, Cl, Br, —C(O)OH, —CH₂—C(O)OH, —C(O)O—CH₃, phosphonate, an optionally partially or fully fluorinated alkyl group (C₁-C₃) or a photoactive group of the formula —Y—Ar—R₅,
Y represents an atom selected from O and S, or an —NH— group,
Ar represents a (C₅-C₁₁) aryl group, and
R⁵ represents a monodentate or bidentate group containing from 1 to 30 carbon atoms.

Even more preferably, the polymer may be selected from:
poly(vinylidene fluoride) homopolymers;
copolymers including at least one unit derived from vinylidene difluoride and at least one unit derived from trifluoroethylene; and
terpolymers including at least one unit derived from polyvinylidene difluoride, at least one unit derived from trifluoroethylene, and at least one unit derived from a monomer selected from hexafluoropropylene, hexafluoroisobutylene, 1-chloro, 1-fluoroethylene, 1-chloro, 2-fluoroethylene, chlorotrifluoroethylene, bromotrifluoroethylene, tetrafluoropropylene, 2-chloro-3, 3,3-trifluoropropene, dimethyl vinylphosphonate, α,β-difluoroacrylic acid, itaconic acid, perfluorobutylethylene, pentafluoropropene, perfluoropropyl vinyl ether, perfluoromethyl vinyl ether, 2-trifluoromethacrylic acid.

Of the polymers described above, co-polymers and ter-polymers are particularly preferred.

The piezoelectric polymer(s) suitable for the invention may also be functionalized, i.e. comprise at least one functional group. The functional group may be grafted at the end of the chain of the piezoelectric polymer, i.e. be a terminal functional group, or be grafted to one or more constituent units of the piezoelectric polymer.

The choice of a functional group to be incorporated into the piezoelectric polymer is a matter for the person skilled in the art, depending on the properties to be imparted to said polymer. For example, the functional group can be used to modulate the decomposition or stability of the polymer with a view to its subsequent recycling, to make the polymer sensitive to IR, visible or ultraviolet radiation, to modify the surface energy of the polymer in order to make it more hydrophilic or, on the contrary, more hydrophobic, to modulate the surface hardness of the polymer, to optimize coupling or adhesion to other materials, to modify the viscosity in solution . . .

The functional group(s) may comprise at least one alcohol, acetate, vinyl, azide, amine, carboxylic acid, (meth)acrylate, epoxide, cyclocarbonate, alkoxysilane or vinyl ether function. In particular, the functional group(s) may be selected from —(CH)₂ₙ—OH, —(CH)₂ₙOAc, —(CH)₂ₙ—O—C(O)—CH═CH₂, —(CH)₂ₙ—O—C(O)—C(CH₃)═CH₂, —(CH)₂ₙ—NH₂, —(CH)₂ₙ—C(O)OH, —(CH₂ₙ)—CH═CH₂, —O—CH═CH₂, Si(R)ₘ(OR)₃₋ₘ, —O—CH₂-epoxide and —O—CH₂-cyclocarbonate, with n being an integer from 0 to 10, Ac is an acetate function, m being an integer from 1 to 3, each R representing a (C₁-C₆)alkyl group.

Other examples of functional groups are R⁴ and R⁵.

Piezoelectric polymers suitable for the invention can be synthesized by any method known to the skilled person. Examples of such polymers and their synthesis methods are described, in particular, in documents WO2017/068276, WO2013/160621, WO2017/051109, WO2018/065306, WO2016/055712, WO2017/021783, WO2009/147030, U.S. Pat. No. 6,355,749B1 and WO 2020/128265.

It is understood that these polymers and methods are cited as examples of piezoelectric polymers, and their methods of preparation, that may be suitable for the present invention, without being limiting of the scope of the invention.

In yet another embodiment, the piezoelectric material of the transducers can be a composite material, i.e. a material comprising a polymeric matrix in which particles of piezoelectric material are dispersed.

Preferably, the composite material suitable for the piezoelectric transducer is formed:
a polymeric matrix comprising at least one polymer chosen from a polyethylene terephthalate (PET), a polyethylene naphthalate (PEN), a polyvinyl chloride (PVC), a polyvinylidene chloride (PVDC), a poly(p-xylylene) (parylene), a silicone, a polysulfone, a fluoropolymer such as polytetrafluoroethylene (PTFE), a polyimide, or a polymer comprising at least one unit derived from vinylidene difluoride as defined above; ethylenechlorotrifluoroethylene (ECTFE), ethylene-tetrafluoroethylene (ETFE) or ethylene-perfluoropropylene (FEP) copolymers, or polychlorotrifluoroethylene (PTFCE), a polyimide, a polyurethane (PU), a polycarbonate (PC), a polyester, polyetherketone (PEEK), polymethylpentene (PMP), polyphenylene sulfide (PPS), polyamide, such as polyamide 6-6 (nylon), polyamide-imide (PAI), polylactic acid (PLA), and copolymers of at least two of the polymers defined above;
and
piezoelectric particles dispersed in the polymeric matrix, said particles consisting of at least one ceramic selected from a lead titanate zirconate (PZT), in particular PZT-4 or PZT-5H, a modified lead titanate (Pb, Ca)TiO3, (Pb, Sm)TiO3, (Ba, Sr) TiO3 (BST), a barium titanate BaTiO3 or a potassium sodium niobate (KNN).

The polymer(s) suitable as a polymeric matrix can be chosen from thermoplastic or thermoset polymers.

The advantage of choosing a thermoset polymer is that it can be molded cold or at low temperature, usually below 80° C.

The polymeric matrix can include any additive customary in the field of polymeric materials, in particular chosen from plasticizers, stabilizers, colorants, etc.

The choice of piezoelectric particles in the composite material is a matter for the person skilled in the art. In particular, the size and loading ratio of piezoelectric particles within the composite can be adjusted to ensure sufficient piezoelectric activity within the transducers.

In particular, piezoelectric particles can be present in the composite material in a proportion ranging from 1 to 99% by mass, in particular from 50 to 80% by mass, relative to the total mass of the composite material.

According to an advantageous embodiment, the dielectric material of the capacitive transducers can be: any piezoelectric material, as described above, as well as cross-linked PE, PP, PVC, PTFE, epoxy resin, polycarbonate, ceramics, Al₂O₃, SiO₂, AlN, TiO₂, and any composite polymer formed from these materials.

The layer(s) of the device comprising the piezoelectric or capacitive transducers can be made to conform to the part of said human or animal body against which the substrate is applied.

For example, piezoelectric or capacitive transducers are advantageously made of a flexible material.

Piezoelectric polymers and composites are particularly suitable for this purpose.

Thus, a piezoelectric material suitable for implementing the invention is more particularly a material with a Young's modulus less than or equal to 3.0 GPa, in particular ranging from 0.05 to 1.5 GPa, especially from 0.3 to 1.5 GPa.

Advantageously, the dielectric material has a dielectric constant (relative permittivity) greater than 2, preferably from 10 to 2000.

Advantageously, the piezoelectric material has a piezoelectric coefficient (usually designated by the acronym d₃₁ or d₃₃) greater than or equal to 4 pC/N, preferably ranging from 10 to 3000 pC/N.

As previously mentioned, the plurality of piezoelectric and/or capacitive transducers is supported by the substrate defined above.

The piezoelectric transducers can be deposited on the substrate using any deposition technique known to the skilled worker, in particular a solvent-based technique selected from spin-coating, squeegee deposition, blade-coating and ultrasonic spray deposition, slot-die coating, dip-coating, bar-coating, curtain coating, inkjet printing, rotogravure, flexography, lithography and screen printing.

Polymeric piezoelectric materials as defined above are particularly suitable for solvent deposition, and in particular for deposition by inkjet printing, for deposition of the continuous layer required in lithography and for screen printing.

Inks comprising piezoelectric polymers suitable for the invention are described in particular in documents WO2018/215341, WO2019/029975 and WO2020/070419. It is understood that these documents are cited as examples of piezoelectric polymer formulations that may be suitable for the present invention, without limiting of the scope of the invention.

Capacitive transducers can be produced using the materials described in [1].

### Electronic Switching Circuit

As mentioned above, a device according to the invention comprises at least one electronic switching circuit, of the matrix switch type, supported by the substrate or by a support fixed to the substrate, and whose inputs are intended to be supplied with ultrasound signals, possibly of different phases, the outputs are each connected to one of the piezoelectric or capacitive transducers and the control bus is intended to be connected to at least one command processor.

Preferably, each group is activated individually by a matrix switch.

In general, each piezoelectric transducer conforming to the invention can be individually addressed, and a distribution of electrical signals distributed over the various transducers of a group is produced which focuses the ultrasound signals produced in the manner of a phased array ultrasound antenna. The main lobe of the resulting focused ultrasonic signal can be oriented at 0° from the normal to the transducer's emitting surface, with the possibility of the antenna formed scanning up to a high angle, typically up to 30° from the above normal, thus enabling the detection of the desired target when its location in the human or animal body to be investigated is not initially known.

### Multi-Group Mode

According to an advantageous embodiment, the device comprises:
a plurality of groups of piezoelectric transducers supported by the substrate;
a plurality of matrix switches supported by the substrate, the inputs of the piezoelectric and/or capacitive transducers of each group being connected to at least one matrix switch.

According to another particular implementation, the device can be made with different matrix switches connected in parallel to groups of transducers. They can be activated by programming, and/or by activating the corresponding common electrodes.

Preferably, the groups are evenly distributed over the substrate surface.

In an advantageous variant, the device includes at least one matrix switch type electronic control circuit, connected to at least some of the transducer groups.

In another advantageous variant, the device comprises, for each piezoelectric transducer, a stack of actuation electrode/transducer piezoelectric material/actuation electrode, deposited directly or indirectly on the substrate.

Advantageously, the device comprises at least one planarization layer based on an electrically insulating material, between the stack and the substrate and/or above the stack.

Advantageously still, the underlying actuation electrode or the overlying one is common to all the plurality of transducers for a given group, this common electrode being connected to a common matrix switch by means of at least one electrically conductive track, the common matrix switch being attached directly or indirectly to the substrate.

Advantageously, the device comprises at least one electromagnetic shielding layer between the stack and the substrate, or on the side of the substrate opposite the stack, optionally coated with a layer of electrically insulating material.

### Interface Layer

The device according to the invention may comprise an interface layer above the stack, intended to be in direct contact with the human or animal body to ensure the transfer of ultrasound signals from and to said body and/or from and to the external device, the interface layer being adapted to conform to the part of said body against which the substrate is applied.

In a preferred embodiment, the interface layer is an acoustic impedance-matching layer.

The interface layer may consist of a gel layer, in particular an ultrasonic transmission gel.

It can be an aqueous gel, or “hydrogel”, i.e. a gel comprising water as the majority solvent.

Ultrasonic gels typically comprise water, a polymer, in particular a thickening polymer, and optionally a humectant. Examples of gels suitable for the invention are described in particular in patent applications EP0347517A1 and FR2395006A1. It is understood that these hydrogels are cited as examples of ultrasonic transmission gels that may be suitable for the present invention, without being limiting of the scope of the invention.

The interface layer can consist of an adhesive layer, i.e. a layer of a material enabling the substrate to adhere to the skin or any part of the body, laminated integrally with the substrate to form an adhesive patch.

It is understood that the adhesive layer, when present, must not hinder the transmission of ultrasonic waves.

Such an adhesive layer may comprise a polymer with adhesive properties, such as polyvinyl alcohol, a styrene/isoprene/styrene copolymer or an acrylate/vinyl acetate copolymer.

In particular, the adhesive layer can be selected from materials conventionally used in adhesive dressings, including hydrogel dressings such as those described in EP1390085B1 or WO2010/067378 A2, or silicone dressings, for example those marketed as Cica-Care® by Smith & Nephew, or from materials conventionally used for the adhesion of transdermal patches, such as those described in EP1676895A1. Nanostructured adhesives may also be suitable as an adhesive layer. It is understood that these materials are cited as examples of adhesives that may be suitable for the present invention, without being limiting of the scope of the invention.

According to this embodiment, a protective peel-off sheet can cover the adhesive layer, to facilitate handling and prevent foreign matter from altering the adhesive properties, before the adhesive patch is applied to the human or animal body. The peelable sheet can be made of any suitable material with low adhesion to facilitate peeling, such as paper or polymer (polyester, polyethylene, ethylene/vinyl acetate), optionally coated with a silicone layer.

### System

Another object of the invention is a system for exchanging electrical energy and/or data, in particular for localization and, where appropriate, tracking purposes with at least one implant in a human or animal body and/or with at least one organ of a human or animal body, such as part of a nerve, and/or with at least one device external to a human or animal body, comprising:
at least one device, as described above;
an electronic control unit comprising:
at least one command processor connected to each programmable unit and each matrix switch of the locating device(s) by a control communication bus(es),
a multiphase ultrasound generator, adapted to generate one or more ultrasound signals, of controlled phase(s), identical or not;
a device for processing ultrasound signals from inside the human or animal body received by the piezoelectric and/or capacitive transducers, comprising a digital and/or analog signal processor.

According to an advantageous embodiment, the multi-phase ultrasound generator comprises:
an ultrasound signal generator, which may be programmable;
a phase shifter, possibly programmable, whose input is connected to the signal generator and whose outputs produce phase-shifted ultrasound signals.

The multi-phase ultrasound generator can also be implemented by a Direct Digital Signal Synthesis (DSS or DDSS or D2S2) device, which operates by directly generating each signal independently.

In another advantageous embodiment, the signal processing device includes a phase correction unit, possibly implemented by a processing unit, which may use the phase shifter of the multi-phase ultrasound generator adapted to operate in bidirectional mode, or another phase shifter which may be programmable.

According to a first control variant, the command processor is configured to control the multiphase ultrasound generator and the control inputs of at least one matrix switch, so that each of the plurality of elementary piezoelectric and/or capacitive transmitters/receivers of a group is supplied for a given duration with signals whose phase is adjusted in order to concentrate the ultrasonic waves emitted from the group in the form of a focused beam at a point P of any Cartesian three-dimensional coordinates, X, Y, Z, or at any elevation and azimuth angles, the point P corresponding to a point on the implant or organ, or to a reference point in the body.

According to a second control variant, the command processor is configured to control the multiphase ultrasound generator and the control inputs of at least three matrix switches, so that each of the plurality of elementary piezoelectric and/or capacitive transmitters/receivers of three distinct groups is sequentially supplied with phase-adjusted signals in order to concentrate the ultrasonic waves emitted from each of the at least three distinct groups into focused beams at a point P and to determine its XYZ coordinates relative to a reference frame by triangulation calculation performed by the signal processing device on the basis of the ultrasound signals received by each of the plurality of elementary piezoelectric transmitters/receivers of a group, as a result of the partial or total reflection of the transmitted ultrasound signals.

Advantageously, the system includes a user alert means, when the determined Cartesian XYZ coordinates of the implant are either within or outside a region defined by reference coordinates, possibly taking tolerances into account.

In another advantageous embodiment, the system includes a device for imaging or displaying the implant's three-dimensional positioning, the operation of which is slaved to the determination of the implant's Cartesian XYZ coordinates.

In general, the physiological effects of the device or implant can be monitored using medical imaging techniques such as functional magnetic resonance imaging (fMRI), positron emission tomography (PET), single photon emission computed tomography (SPECT) or CT scanning. These imaging techniques make it possible to adapt the physiological effects of a device according to the invention or of the implant, and can therefore form part of a system according to the invention, with or without closed-loop or automatic servo-control.

According to this other mode, a user interface is advantageously provided, preferably remote, connected to the imaging device.

In some configurations, the system comprises a number of location and possibly tracking devices, the substrates of which are physically separated.

In this case, the group(s) of piezoelectric transducers of at least one of the devices can be configured to emit ultrasound signals from the outside of the body to the inside, while the group(s) of piezoelectric transducers of at least one other of the devices is (are) configured to receive ultrasound signals from the inside of the body.

To make the system autonomous, it can include a power supply battery.

Preferably, at least one part of the electronic control unit, together with the at least one tracking device, forms a single structure to be applied to the human or animal body.

Even more preferably, the tracking device(s) is (are) connected to the electronic control unit and, where applicable, to a unit for acquiring and/or recording the ultrasound signals received and, where applicable, to a monitoring and/or warning unit and, where applicable, to the battery, the device(s) and the connected unit(s) being housed in an autonomous case to be worn by the human or animal.

Advantageously, the autonomous case is configured to be connected by wired connection or by a wireless communication protocol such as wifi, NFC or Bluetooth, to an external computing or imaging system.

In general, for P target recognition, a proven technology can be used, such as that described in publication [2].

In general, for real-time imaging, we can implement already proven imaging with 3D image reconstruction and visualization in the field of ultrasound, as for example disclosed in patent U.S. Pat. No. 5,787,889A.

Diagnostic and/or Therapeutic Method

The invention also relates to a diagnostic and/or therapeutic method implemented by at least one system just described.

More particularly, the method comprises the following steps:
a/emission of a plurality of ultrasound signals by at least one group of a plurality of piezoelectric and/or capacitive transducers of a device;
b/reception by at least one group of a plurality of piezoelectric and/or capacitive transducers of a device of ultrasound signals coming from the implant and/or organ and those coming from the interior or exterior surface of the human or animal body;
c/digital processing of ultrasound signals to locate the implant and/or organ;
d/if necessary, the implant emits other signals autonomously or in response to the signals emitted according to step a/and/or performs local actions in or on the body, such as electrical, mechanical 1 or chemical 1 activation, displacement, change of shape, release or deposition of an active ingredient, marker or other material.

### Medical Kit

The invention also relates to a medical kit comprising:
a system as described above;
an implant to be placed in or on a human or animal body.

### Implant

The object forming the implant in the sense of the invention can be extracted from the human or animal body, set in motion, i.e. voluntarily moved within or on the body.

Preferably, at least part of the implant's outer surface is structured to enhance the reflection of ultrasound signals emitted by the piezoelectric transducer array(s). An implant material can be selected or structured to optimize the reflection of ultrasound signals within the human or animal body in which the implant is present. This ensures greater precision.

Preferably, the implant material may have undergone an external surface treatment for catadioptric reflection, which is all the more advantageous for implant surfaces of small or very small dimensions. The surface treatment may consist of surface micro-structuring with patterns/textures of 10 to 500 μm.

When the implant is intended to provide stimulation, it can itself generate ultrasound waves that will generate physical contact/pressure only. The implant may also directly generate an electrical current through at least one contact capable of exchanging electrical charges, generally via one or more electrodes, in order to establish one or more points of electrical stimulation in the body. This electrical current generation can also drive a chemical reaction or be driven by a chemical reaction. For example, biomarkers can be estimated either by measuring heart rate to quantify the pressure applied, or by measuring pH to quantify the electrical or chemical contact established.

An implant according to the invention can be adapted to receive energy from the device's piezoelectric transducers: to this end, it can comprise one or more piezoelectric energy harvesters (PEH). An implant according to the invention can also be adapted to receive energy directly from a human or animal body, by means of its own vibrations, pressures, heartbeats, etc.

An implant according to the invention can incorporate a processor enabling it, among other things, to pre-process received data and/or communicate with the device according to the invention to transfer data to the latter and receive data from the latter.

The same device or system according to the invention can exchange data and/or energy with a plurality of implants within the same human or animal body. Data and/or energy can also be exchanged between a plurality of implants within the same human or animal body.

### Applications

Generally speaking, the invention can be applied to the treatment of any pathology related to neurology, which concerns:
or the peripheral nervous system, such as obesity, local dermatology, inflammation, metabolism, pain, bladder dysfunction, cardiac disorders, etc.
or the central nervous system, such as cognitive disorders, epilepsy, Alzheimer's disease, Parkinson's disease, headaches, migraines, post-stroke treatment,

Dermatological pathologies may require monitoring and/or local treatment using the invention described in this patent.

The advantages of the invention are numerous, including the following:
non-invasive or minimally invasive neuromodulation to treat neurology-related pathologies, with the possibility of configuring and temporarily replacing a device/system quickly and easily according to the invention;
the ability to precisely reach tissues at any depth in the human or animal body, typically from 1 to 20 cm from the skin;
the ability to activate different types of nerve fibres locally and selectively;
acute/semi-chronic/chronic stimulation (benefiting from the non-invasive paradigm);
highly specific stimulation, provided in part by the spatial precision with which ultrasound waves are focused,
closed-loop stimulation, i.e. adapting diagnosis/therapy to the patient's physiological state or response;
the ability to record physiological parameters or biomarkers, before, during and after stimulation by ultrasound waves emitted by transducers.

Specifically, applications of the invention can be:
monitor the spatial evolution of an implant, such as a tissue patch, a bariatric device, a prosthesis or pacemaker, or at least part of a catheter, probe, introducer or wire guide introduced into the human body;
monitor the kinetics of movement or degradation of an implant as it is evacuated through natural channels, such as the spatio-temporal evolution of a gastrointestinal implant during the digestion process, or the degradation of a biodegradable gastrointestinal device;
monitoring of fiduciary markers, oocytes in a tube, crystals obstructing a duct (kidney stones, gallstones);
determine and/or realign the positioning of invasive devices during surgical operations, such as biopsy or tumor removal, or during epidural anesthesia to determine the location of the needle relative to the environment;
extract physiological data from a human or animal body;
determine and/or readjust the positioning of a device for delivering one or more drugs or markers, tracers or materials in the human or animal body;
activate and/or control a device inside the implant, possibly in a closed loop, depending on measurements made in situ by the implant and/or depending on other parameters;
activate a device for stimulating a peripheral nerve, such as a vagus nerve, when a peristaltic movement is detected in the digestive tract of a human body.

Generally speaking, the device in the form of a patch conforming to the invention can be single-use or reusable. It can be removed, moved with a patient, used to monitor the evolution of a tissue, an organ or an implant.

A patch according to the invention can be arranged outside a patient's skin, directly on the skin, subcutaneously, on a mucous membrane, in a natural cavity, for example in a colon, esophagus, vagina, etc., introduced intravascularly, in the lymphatic system, on elements outside a human or animal body such as an object or an individual other than the patient whose organ is to be located or whose body receives an implant to be located.

With a system based on the invention, geolocation can be envisaged between two distinct physical entities, for example between a patient or one of his anatomical parts and a technician in charge of the system, or between a patient and an assistance and/or intervention robot.

We can also envisage communication with any type of connected object.

We can also envisage communication between two or more separate implants, with one acting as “master” and the other(s) as “slave”.

As a support, one or more patches according to the invention can be integrated into a helmet frame or a garment, or an accessory such as a belt, a bandage, a shoe, a glove, a sheath, or even furniture such as a chair, an armchair, a bed, a medical device, etc.

Further advantages and features will become apparent from the detailed description, which is illustrative and non-limiting, with reference to the following figures.

### BRIEF DESCRIPTION OF THE DRAWINGS

FIG. 1 is a schematic perspective view illustrating the main elements of a device for locating and, where appropriate, tracking an implant in a human or animal body or an organ of a human or animal body, according to the invention, the device being in the form of a patch.

FIG. 2 is a schematic front view showing the groups of piezoelectric transducers with their matrix switches of a patch according to the invention.

FIG. 3 is a longitudinal sectional view partially illustrating an example of a patch according to the invention, produced by a multilayer printed circuit technique or by the deposition of thin layers on a flexible, conformable substrate.

FIG. 4 is a schematic front view showing the groups of piezoelectric transducers with their matrix switches in a patch as shown in FIG. 3.

FIG. 5 is a block definition diagram of a localization and tracking system according to the invention comprising a patch according to one of FIGS. 1 to 4, an electronic control unit, an imaging or display device and a monitoring and alerting unit.

FIG. 6 schematically illustrates the transmission of ultrasound signals by groups of piezoelectric transducers of a device according to the invention, operating as an ultrasound phased array antenna, to a target (organ, implant) within a human or animal body.

FIG. 7 shows a schematic front view of the location and tracking of a moving target (organ, implant) within a human or animal body, using a system based on the invention and based on triangulation calculations.

### DETAILED DESCRIPTION

The various elements shown in the figures are not to scale for the sake of clarity.

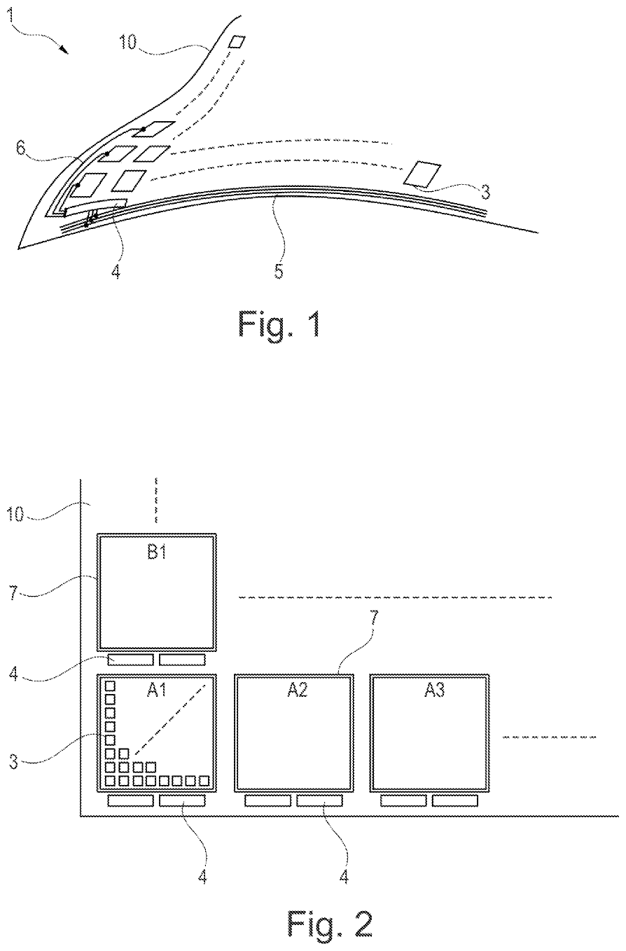

FIGS. 1 and 2 show an example of a device for locating and tracking an implant in a human or animal body, in the form of a patch 1 to be applied, preferably by adhesive, to the body.

The whole of patch 1 is flexible and conformable to any part of the body's anatomy.

The patch 1 comprises a substrate 10 made of an electrically insulating polymer, such as polyimide.

The substrate 1 supports a plurality of groups A1, A2, A3 . . . ; B1 . . . each comprising a plurality of elementary PVDF piezoelectric transducers 3. Each transducer 3 can be a discriminating zone of a flexible PVDF film or layer, of small thickness, typically of the order of 100 μm.

As explained below, each transducer 3 forms an elementary piezoelectric transmitter/receiver configured to transmit ultrasound signals from the outside of the human or animal body to the inside and/or to receive other ultrasound signals, including those from the inside of the body by reflection, echo, etc.

Typically, each group A1, A2, A3 . . . ; B1 . . . may comprise a number of 8×8, 16×16 or 32×32 elementary transducers 3.

The substrate 1 also supports a plurality of matrix switches 4, the piezoelectric transducer inputs of each piezoelectric group being connected to at least one matrix switch-type electronic switching circuit by at least one conductive signal distribution line 5, to produce ultrasound.

More precisely, as detailed below, a conduction line 6 feeds an actuation electrode of the piezoelectric material of an elementary transducer 3. An actuation electrode 7 may be common to all transducers 3 of a group as shown in FIG. 2 and be connected to a common matrix switch.

As also shown in FIG. 1, the inputs of a matrix switch 4 are intended to be supplied with ultrasonic signals, possibly of different phases 5. The matrix switch is further connected to a command processor as detailed below.

Each matrix switch 4 is an integrated circuit, most commonly an ASIC (Application Specific Integrated Circuit), possibly of the LSI (Large Scale Integration) type. Typical formats range from qqs to over 100 mm².

The integration, i.e. the support and interconnection of the plurality of transducers 3 and matrix switches 4 on a flexible and conformable substrate 10 in order to constitute a patch 1 according to the invention, can be done by a multilayer printed circuit type technology and/or by thin film deposition.

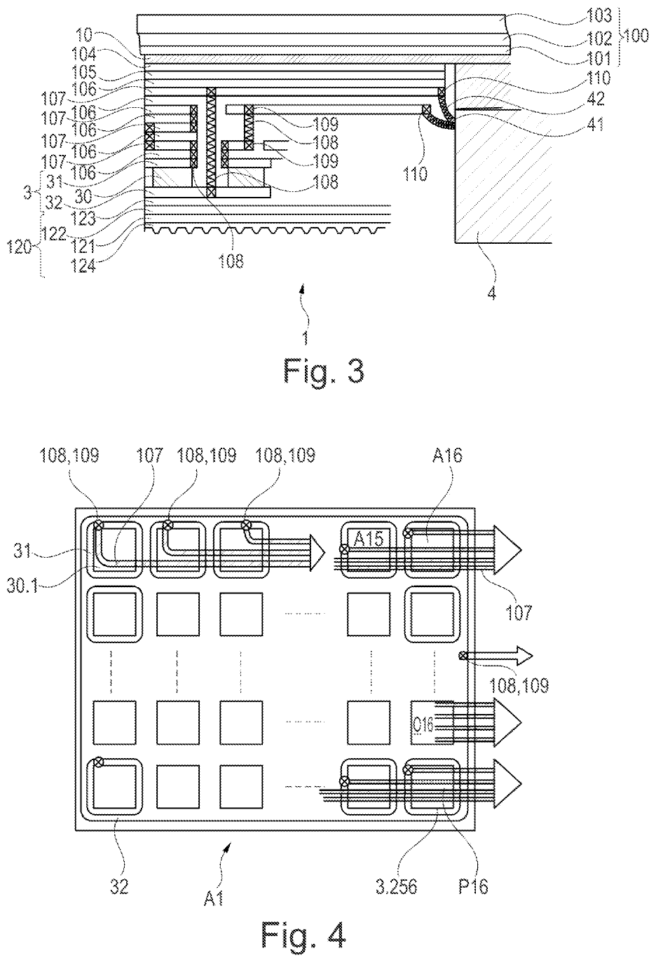

An example of this integration is shown in FIGS. 3 and 4 in the form of a stack of functional layers linked to the integrated circuits forming the matrix switches 4.

The substrate 10 first supports an overlying subassembly 100 for protection from the external environment.

This overlying subassembly 100 is a stack of three layers, namely:
an inner layer 101, directly in contact with the substrate, forming an electromagnetic shielding against external signals. This 101 layer can be a metallic layer of Cu, Ag or Au, or a layer of PEDOT:PSS or PEDOT:PSS/IL (doped with ionic liquid). Where appropriate, the layer 101 has an additional mechanical protection function. For this purpose, the appropriate mechanical characteristics are selected;
an intermediate layer 102 forming a mechanical screen, which may be in the form of a high-density film, to optimize ultrasound signal emission towards the opposite side of substrate 10. This layer 102 may be made of a polymer containing BaSO₄ and/or TiO₂ particles;
an outer layer 103, directly in contact with the external environment, forming a chemical protection resistant to solvents, alcohols, adhesives, etc. This layer 103 can be a flexible polymer film.

The substrate 10 also directly supports the integrated circuits 4 forming the matrix switches, which can be glued or printed directly onto the substrate 10.

A planarization layer 104 is in direct contact with the side of the substrate 10 opposite the side supporting the protective assembly 100. The function of layer 101 is to facilitate deposition of the other layers. This planarization layer 104 may be that of a pre-planarized PEN-type polymer film (e.g. Teonex® planarized polyethylene naphthalate).

Directly underlying the planarization layer 104 is an electrically conductive layer forming a ground conductor 105. This layer 105 can be a metallic layer of Cu, Ag or Au, or a layer of PEDOT:PSS or PEDOT:PSS/IL (doped with ionic liquid).

Underneath are alternating electrically insulating layers 106 and conductive layers forming intermediate electrodes 107 for electrical interconnections with the piezoelectric transducers 3.

The layers 106 are either low-dielectric insulators of the SiO₂ type, in particular those obtained by polycondensation of silanes, for example (TEOS), or insulating oxide layers or polymers, in particular those based on Si. The electrically insulating layers 106 can be produced by deposition or can consist of an electrically insulating film coated on both sides with an adhesive for integration into the stack.

Intermediate electrodes 107 may be identical to layers 105.

More precisely, each transducer 3 comprises a discrete zone 30 of piezoelectric material and two discrete zones 31, 32 of actuation electrodes on either side of the piezoelectric material 30. Zones 31, 32 can advantageously be made of an electrically conductive polymer to optimize the acoustic impedance of transducers 3.

As shown in FIG. 4, the actuation electrode 32 can be a continuous layer, common to all transducers 3 in a group.

An electrode 31 can be a metallic layer of Cu, Ag or Au or, more incidentally, a layer of ITO or PEDOT:PSS or PEDOT:PSS/IL (doped with ionic liquid).

An electrode 32 can be made of an electrically conductive polymer, such as PEDDOT:PSS or PEDOT:PSS:IL (doped with ionic liquid), etc., which provide optimized acoustic impedance.

Electrical interconnections 109 across the stack between the intermediate electrode layers 107 themselves and with the actuation electrodes 31, 32 are made by means of electrically conductive vias 108, typically of copper or tungsten.

Electrical interconnections 110 between intermediate electrode layers 107 and an integrated circuit 4 are made by means of connection pins 41, 42, whose contacts are soldered or brazed, for example by solder balls or solder bumps.

The substrate 10 also supports, on the side of the transducers 3 intended to be applied against the human or animal body, an underlying interface subassembly 120.

This underlying subassembly 120 is a stack of one to four layers, namely:
an electrically insulating layer 121 which may be in contact with the actuation electrode 32 of transducer 3, in the absence of layers 122 and 123. This layer 121 may be a polymer film, of PVDF or ECTFE for example, or of optimized acoustic impedance;
an optional electrically conductive layer forming a ground conductor 122 directly in contact with layer 121, whose function is to provide electromagnetic shielding against signals/disturbances generated from the human or animal body. This layer 122 can be made of the same material as layer 32;
an optional planarization layer 123 in direct contact with layer 121 or 122. This layer 123 is intended to fill the cavities associated with the integration of elements 30 to 110, and can be made of any flexible, electrically insulating polymer with acceptable acoustic impedance, preferably a flexible, elastic thermoset polymer, which can be cold or low-temperature cast, generally below 80° C.;
an optional interface layer 124 based on gel and/or adhesive and/or structured adhesive with micro- or nanoscale patterns for direct contact with the human or animal body.

This interface layer 124 is adapted to best conform to the relief of the human or animal body.

Preferably, at least one or all of the layers 121, 122, 123, 124 are designed to optimize acoustic impedance for the most efficient transmission of ultrasonic waves.

It goes without saying that in the integration just described on a substrate 10, the only elements that are imperative are the layers 30, 31, 32 forming a transducer 3, the integrated circuit 4 and its connections 41, 42 to the actuation electrode zones 31, 32 of the transducers. All the other advantageous layers described are optional.

Additional planarization layers such as layer 104 can also be deposited between two layers of electrical insulation.

For example, transducers 3 with surface dimensions equal to 200×200 μm, a 50 μm pitch between actuation electrode zones 31, 32 of two adjacent transducers 3, electrode conduction tracks 31, 32 30 μm wide, separated from each other by 20 μm. In the end, with a number of 100 groups of transducers 3 made with the aforementioned values, i.e. 8×8 mm each, a patch 1 with total surface dimensions equal to 80×80 mm can be dimensioned.

In the case of a subgroup consisting of a number of 16×16 transducers, i.e. 256 transducers, four independent layers of tracks 31, 32 are required for a number of 4 matrix switches, arranged separately on at least two edges of a group A1, A2 . . . . Tracks 107 connect each of the 256 transducers to its matrix switch 4, as shown in FIG. 4.

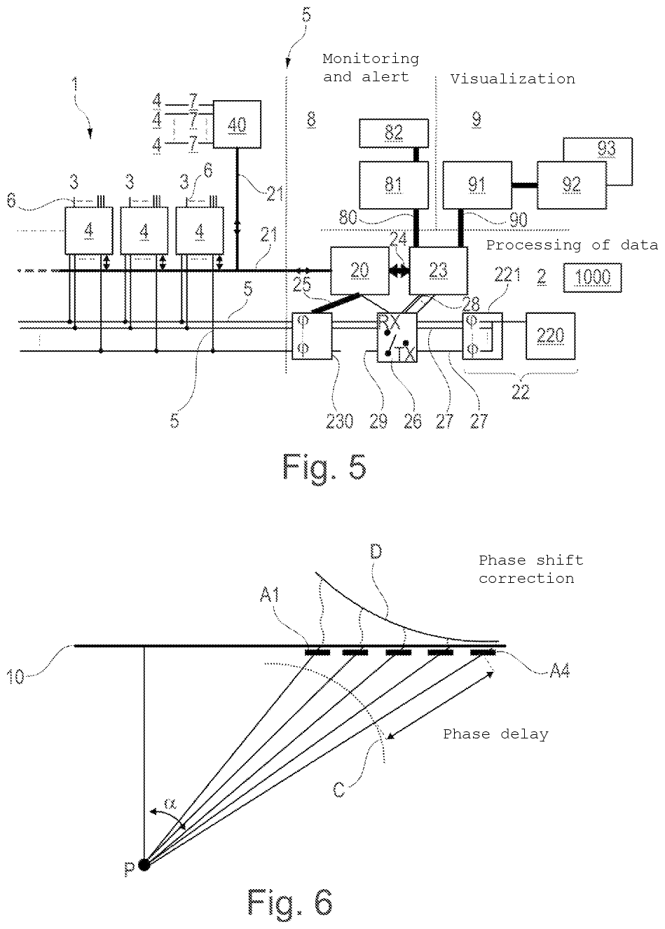

FIG. 5 shows the structure of a complete tracking and tracing system(S) incorporating at least one device in the form of a patch 1 just described.

Each patch 1 may also include switching circuits 40 common to several matrix switches 4, responsible for sequentially addressing group electrodes 7 and activating the corresponding circuits 4.

Alternatively, the matrix switch can integrate the functional part corresponding to circuit 40 and thus take charge of its own activation and the activation of common electrode 7.

The system S comprises, in addition to a patch 1, an electronic control unit 2 comprising:
a command processor 20 connected to one or more programmable units 23 and to one or more matrix switches 4 of a patch 1 by a control communication bus(es) 21, typically a 2- or 4-wire high-speed serial bus;
a multiphase ultrasound generator 22 adapted to generate one or more ultrasound signals, of controlled phase(s), identical or not;
a device for processing ultrasound signals from inside the human or animal body picked up by the piezoelectric transducers 3, comprising a digital and/or analog signal processor 23 which communicates with the command processor 20 via control bus(es) 24 and with the phase shifter 230 (optional) via bus 25.

As shown in FIG. 5, the multi-phase ultrasound generator comprises:
an ultrasonic signal generator 220, which may be programmable;
a phase shifter 221, possibly programmable, whose input is connected to the signal generator and whose outputs produce phase-shifted ultrasound signals.
or
a DDSS processor.

The signal processing device includes a phase correction unit 230 connected to the command processor via bus(es) 25, optionally controlled by processor 23 or 20.

As shown in FIG. 5, this unit 230 may use a phase shifter identical to the phase shifter 221 of the multi-phase ultrasound generator, adapted to operate in receive-only mode. The processed signals are then sent by buses 28 and 29, via a transmit/receive switch 26 (or any equivalent one-way device such as a circulator) to processor 23.

Switch 26 thus enables ultrasound signals to be routed either for transmission (Tx) from ultrasonic generator 220 via buses 27 to the matrix switches, or for reception (Rx) from matrix switches 4 to digital processor 23 via communication buses 28.

We'll now explain how the S system works.

The command processor 20 is configured to control the multiphase ultrasound generator 22 and the control inputs of the matrix switches 4 so that each group of elementary piezoelectric transducers 3 functions as a kind of phased array ultrasound antenna.

In this way, the commands of the command processor 20 enable each piezoelectric transducer 3 to be supplied with phase-adjusted signals for a set period of time, in order to concentrate the ultrasonic waves emitted from a given group in the form of a focused beam at a point P of any Cartesian three-dimensional coordinates, X, Y, Z, or at any elevation and azimuth angles, the point P (target) corresponding to a point on the implant or organ, or to a reference point in the body.

This focusing is shown in FIG. 6: each group A1 . . . A4 of transducers emits a focused beam on point P, the angle α designating the position of target P relative to the center of the group. In ultrasound signal processing, an electronic phase shift correction, symbolized by the line D, is applied to take into account the phase delay with respect to the constant-phase front circle, symbolized by the circle C (spherical) and centered on point P, and/or the variable phase delay due to the variable acoustic impedance and/or the variable length of the space travelled by the ultrasound wave between the transducer and the implant, for example in the case where the patient is not immobilized.

To determine the tracking of a moving target P, which may correspond to unwanted movement or deformation of an implant in a human or animal body, the command processor 20 controls the multiphase ultrasound generator 22 and the control inputs of at least three matrix switches 4 to achieve focusing from three distinct groups of piezoelectric transducers.

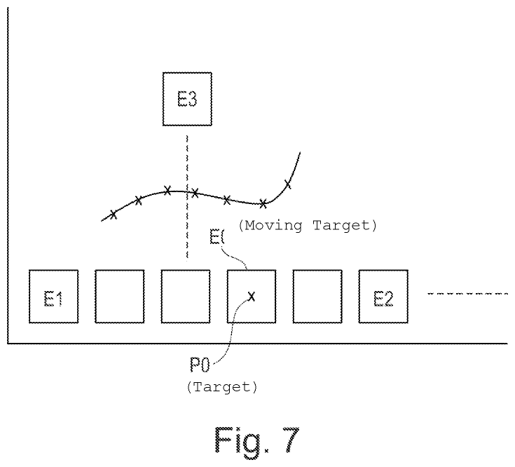

This configuration is shown in FIG. 7. The groups E1, E2, E3 that are chosen for control are those such that their respective angles α1, α2, α3 are the maximums for expanding the total coverage area of the three groups.

The command processor 20 sequentially feeds these three groups E1, E2, E3 with phase-adjusted signals to concentrate the ultrasound waves emitted by each of the three distinct groups E1, E2, E3 into focused beams at a point P1.

A triangulation calculation by the digital processing unit 23 enables the XYZ coordinates to be determined with respect to a reference frame on the basis of the ultrasound signals received by each of the plurality of elementary piezoelectric transmitters/receivers of a group E1, E2 or E3, following the partial or total reflection of the ultrasound signals emitted.

Note that when a P0 target is at shallow depths, i.e. less than half the size of a given transducer group E0, the position and tracking of the P0 target can be obtained without triangulation, but directly by focusing the E0 group's beam alone.

The piezoelectric transducers can be addressed by the command processor 20 in a single-frequency scaling mode or in multifrequency scaling modes.

The first single scale corresponds to the case where the command processor 20 addresses all groups A1, A2 . . . of transducers 3 with a single fixed frequency f as required for a given resolution triangulation calculation. For example, with a number of 100 transducer groups, addressing at a fixed frequency f and the resulting triangulation calculation will be carried out with at least three separate groups. The advantage of this single-scale mode is that it provides maximum accuracy and is simple to implement.

A second scale corresponds to signals addressed with frequency f/2 to at least four distinct groups of 1024 transducers: the command processor 20 usually addresses, in the case of the single scale with frequency f above, an ultrasound signal of given phase to each transducer 3 individually. In this f/2 frequency mode, it transmits the same given phase signal to four adjacent transducers 3 squared with frequency f/2. These four transducers then function as a single transducer (“macrotransducer”) of size 4, the four groups then functioning as a single group (“macro-group”) of 1024 transducers, addressed by a single matrix switch or by four matrix switches 4 operating with a common control.

A third scale corresponds to signals addressed with a frequency f/3, and with a number of nine transducers per macro-transducer and nine groups per macro-group.

A fourth scale corresponds to signals addressed with a frequency of f/4, and with a number of sixteen transducers per macro-transducer and sixteen groups per macro-group.

An n^{th} scale corresponds to signals addressed with a frequency f/n, and with a number of n² transducers per macro-transducer and n² groups per macro-group.

For example, a tenth scale corresponds to signals addressed with a frequency f/10, and with a number of one hundred transducers per macro-transducer and one hundred groups per macrogroup.

All modes other than the single-scale mode provide increased localization coverage, higher transmitting power and therefore greater sensitivity and flexibility, with the added option of reducing the control electronics to larger sizes (more than 100 groups, more than 80×80 mm),

The system S may further comprise a monitoring and alerting unit 8 directly connected to the digital signal processor 23 via a communication bus 80. This unit 8 may comprise monitoring and positioning means 81, and user alert means 82. The alert can be a sound signal, a visual signal, etc. Thus, when the determined Cartesian XYZ coordinates of the implant are either within or outside a region defined by reference coordinates, if necessary considering tolerances, the alert device can send a signal or message to the user.

Finally, the S system includes a display unit 9 directly connected to the digital signal processor 23 via a communication bus 90. This unit 9 may include a device 91 for imaging or displaying the implant's three-dimensional positioning, the operation of which is slaved to the determination of the implant's Cartesian XYZ coordinates.

In system operation, the programmable units 23 ensure in particular:
coordinating the command processor(s) 20;
processing and interpreting signals from bus 28 to extract data of interest, such as implant coordinates;
storage/archiving of data and results so that they can be transmitted as required
transmissions, in particular to and from monitoring and positioning means 81 and imaging device 91.

A wired user interface 92 or a remote interface 93, connected to the imaging device, enables real-time visualization of the implant and its positioning.

Finally, at least one power supply battery (1000) can be provided for the electronic control unit 2, to make the 1 and 2 assembly autonomous in terms of its power source, and therefore portable.

Other variants and improvements can be envisaged without going beyond the scope of the invention.

While the transducers in the illustrated embodiment are piezoelectric, capacitive transducers, or even a combination of the two, can be used in the same device according to the invention.

While the triangulation calculation in the illustrated example is based on three separate groups, it may be possible to use a larger number of groups, in particular to increase localization accuracy. For example, the triangulation could be carried out using four or six separate groups.

A device or system for locating and, where appropriate, monitoring an implant according to the invention can be combined with any device/equipment using one of the various existing non-invasive medical imaging techniques (X-ray, MRI, conventional ultrasound, PET). Among other benefits, this complementary combination can save diagnosis and/or intervention time. For example, a conventional technique can be used to register the initial detection to be performed by the transducers according to the invention in a specific zone of interest and/or to avoid having to initially define reference points.

List of references cited
[1]: C. Domingues, “Design of micromachined acoustic transducers”, https://tel.archives-ouvertes.fr/tel-00009846/document, Jul. 28, 2005.
[2]: Inggs, M. R.; Robinson, A. D., “Ship target recognition using low resolution radar and neural networks”; (1999) IEEE Transactions on Aerospace and Electronic Systems, volume 35, issue 2, pages 386-393, April 1999.

1-47. (canceled)

48. A device for exchanging at least one of energy or data, in particular for localization and, where appropriate, tracking purposes, with at least one selected from:
(a) at least one implant in or on, at least one of a human or animal body, or at least one organ of a human or animal body, such as part of a nerve; or
(b) at least one apparatus external to a human or animal body, the device comprising:
a substrate to be applied preferably against the body from the outside;
at least one group of a plurality of transducers selected from the group of piezoelectric transducers or capacitive transducers, supported by the substrate,
wherein each transducer forms an elementary piezoelectric or capacitive transmitter/receiver configured to perform at least one selected from the group of:
(i) emit ultrasound signals from at least one selected from the outside or inside of the body, to at least one selected from the inside or the outside surface of the body, or to the external apparatus; or
(ii) receive other ultrasound signals including those from at least one selected from the inside of the body or the external apparatus;
at least one electronic switching circuit, of the matrix switch type, supported by the substrate or by a support fixed to the substrate, whose inputs are designed to be supplied with ultrasound signals, possibly of different phases, the outputs each being connected to one selected from the group of the piezoelectric or capacitive transducers, and whose control bus(es) are intended to be connected to at least one command processor device in which at least one selected from the group of the piezoelectric or capacitive transducers are configured to operate as a phased array ultrasound antenna.

49. The device as claimed in claim 48, the substrate consisting of one or more layers of at least one selected from the group of a flexible or stretchable polymer film selected from polyethylene terephthalate (PET), polyethylene naphthalate (PEN), polyvinyl chloride (PVC), polyvinylidene chloride (PVDC), poly(pxylylene) (parylene), silicone, a polysulfone, a fluoropolymer such as polytetrafluoroethylene (PTFE), or a polyimide, optionally filled, for example with an inorganic filler, in particular selected from silica, aluminum hydroxide, aluminum oxide, magnesium oxide, barium sulfate, magnesium carbonate and barium carbonate.

50. The device according to claim 48, the piezoelectric material of the transducers being a ceramic selected from a lead titanate zirconate (PZT), in particular PZT-4 or PZT-5H, a modified lead titanate (Pb, Ca)TiO3, (Pb, Sm)TiO3, (Ba, Sr)TiO3 (BST), a barium titanate BaTiO3 or a potassium sodium niobate (KNN) or being a polymer including at least one unit derived from vinylidene difluoride (PVDF).

51. The device according to claim 50, said polymer comprising at least one unit derived from vinylidene difluoride and at least one unit of formula (I), distinct from the unit derived from vinylidene difluoride:
—CR¹R²—CR³R⁴— (I)
formula (I) in which:
R¹ and R² independently of one another represent H, F or an optionally partially or fully fluorinated (C₁-C₃)alkyl group;
R³ represents F, Cl, Br, I, —COOH, —CH₂—COOH, —COO—CH₃, —SOOX, Si(OR)₃, phosphonate or an optionally partially or fully fluorinated (C₁-C₃)alkyl group;
R⁴ represents F, Cl, Br, I, —COOH, —CH₂—COOH, —COO—CH₃, —SOOX, Si(OR)₃, phosphonate, an optionally partially or fully fluorinated (C₁-C₃)alkyl group, or a photoactive group of formula —Y—Ar—R⁵,
X represents F, —OK, —ONa, —OH or —Si(OR)₃,
R represents a methyl, ethyl or isopropyl group,
Y represents an atom selected from O and S, or an —NH— group,
Ar represents a (C₅-C₁₁) aryl group, and
R⁵ represents a monodentate or bidentate group containing from 1 to 30 carbon atoms.

52. The device according to claim 51, the piezoelectric material of the transducers being a composite formed of:
a polymeric matrix comprising at least one polymer selected from polyethylene terephthalate (PET), polyethylene terephthalate (PEN), polyvinyl chloride (PVC), polyvinylidene chloride (PVDC), a poly(p-xylylene) (parylene), a silicone, a polysulfone, a fluoropolymer such as polytetrafluoroethylene (PTFE), a polyimide, or the polymer polymer comprising the at least one unit derived from vinylidene difluoride and the at least one unit of the formula (I), distinct from the unit derived from vinylidene difluoride; ethylene-chlorotrifluoroethylene (ECTFE), ethylene-tetrafluoroethylene (ETFE) or ethylene-perfluoropropylene (FEP) copolymers, or polychlorotrifluoroethylene (PTFCE), a polyimide, a polyurethane (PU), a polycarbonate (PC), a polyester, a polyetherketone (PEEK), polymethylpentene (PMP), polyphenylene sulfide (PPS), polyamide, such as polyamide 6-6 (nylon), polyamide-imide (PAI), polylactic acid (PLA), and copolymers of at least two of the polymers including the at least one unit derived from vinylidene difluoride (PVDF); and
piezoelectric particles dispersed in the polymeric matrix, said particles consisting of at least one ceramic selected from a lead titanate zirconate (PZT), in particular PZT-4 or PZT-5H, a modified lead titanate (Pb,Ca)TiO3, (Pb, Sm)TiO3, (Ba, Sr)TiO3 (BST), a barium titanate BaTiO3 or a potassium sodium niobate (KNN).

53. The device according to claim 48, comprising:
a plurality of groups (A1-A16; B1-Bn . . . ) of transducers selected from the group of piezoelectric transducers (3), capacitive transducers, or a combination thereof, supported by the substrate (10);
a plurality of matrix switches (4) supported by the substrate, the inputs of the transducers selected from the group of piezoelectric or capacitive transducers of each group being connected to at least one matrix switch.

54. The device according to claim 53, comprising at least one electronic control circuit of the matrix switch type, connected to at least some of the transducer groups.

55. The device according to claim 53, comprising, for each piezoelectric transducer, a stack of actuation electrode/transducer piezoelectric material/actuation electrode, deposited directly or indirectly on the substrate.

56. The device according to claim 55, comprising at least one selected from the group of:
at least one planarization layer based on an electrically insulating material, having a position selected from at least one of: between the stack and the substrate or above the stack; or
at least one conductive electromagnetic shielding layer between the stack and the substrate, or on the side of the substrate opposite the stack, optionally coated with a layer of electrically insulating material.

57. The device according to claim 55, comprising an interface layer above the stack, intended to be in direct contact with the human or animal body to ensure the transfer of ultrasound signals from and to at least one selected from the group of said body or the external device, the interface layer being adapted to conform to the part of said body against which the substrate is applied.

58. The device according to claim 57, the interface layer also being at least one selected from the groups of: an acoustic impedance-matching layer or being a gel layer or a layer of adhesive or nanostructured adhesive, laminated integrally with the substrate to form an adhesive patch.

59. A System(S) for exchanging at least one of electrical energy or data, in particular for localization and possibly tracking purposes, with at least one selected from the group of: at least one implant in a human or animal body or at least one organ of a human or animal body, such as part of a nerve; or with at least one device external to a human or animal body, comprising:
at least one device, according to claim 48;
an electronic control unit comprising:
a command processor connected to one or more programmable units and to one or more matrix switches of the locating device(s) by a control communication bus(es),
at least one multiphase ultrasound generator, adapted to generate one or more ultrasound signals, with controlled phase(s), identical or not;
a device for processing ultrasound signals from inside the human or animal body received by at least one selected from the group of: the piezoelectric or capacitive transducers, comprising at least one of a digital or analog signal processor.

60. The system according to claim 59, the multi-phase ultrasound generator comprising:
an ultrasonic signal generator, optionally programmable;
a phase shifter, optionally programmable, whose input is connected to the signal generator and whose outputs produce phase-shifted ultrasound signals; or
a direct digital synthesis device.

61. The system according to claim 59, the signal processing device comprising a phase correction unit, possibly implemented by a processing unit, this unit possibly using the phase shifter of the multi-phase ultrasound generator adapted to operate in bi-directional mode, or a possibly programmable phase shifter.

62. The system according to claim 59, the command processor being configured to control the multiphase ultrasound generator and the control inputs of at least one matrix switch, so that each of the plurality of elementary transducers selected from the group of capacitive or piezoelectric transmitters/receivers of a group is supplied for a given time with signals whose phase is adjusted in order to concentrate the ultrasonic waves emitted from the group in the form of a focused beam at a point P of three-dimensional Cartesian coordinates, X,Y,Z, or at any elevation and azimuth angles, the point P corresponding to a point on the implant or organ, or to a reference point in the body.

63. The system according to claim 59, the command processor being configured to control the multiphase ultrasound generator and the control inputs of at least three matrix switches, so that each of the plurality of elementary transducers selected from the group of piezoelectric or capacitive emitters/receivers of three distinct groups is supplied sequentially with phase-adjusted signals in order to concentrate the ultrasonic waves emitted from each of the at least three distinct groups into beams and to determine its XYZ coordinates relative to a reference frame by triangulation calculation performed by the signal processing device on the basis of the ultrasound signals received by each of the plurality of elementary transducers selected from the group of piezoelectric or capacitive transmitters/receivers of a group, as a result of the partial or total reflection of the transmitted ultrasound signals.

64. The system according to claim 59, comprising one or more of:
user alert means, when the determined Cartesian XYZ coordinates of the implant are either within or outside a region defined by reference coordinates, where appropriate taking tolerances into account;
a device for imaging or displaying the three-dimensional positioning of the implant, the operation of which is slaved to the determination of the Cartesian XYZ coordinates of the implant;
a user interface, preferably remote, connected to the imaging device;
comprising a plurality of devices, the substrates of which are physically separated;
comprising a plurality of devices, the substrates of which are physically separated, and wherein the group(s) of piezoelectric transducers of at least one of the devices being configured to emit ultrasound signals from the outside of the body to the inside, while the group(s) of piezoelectric transducers of at least one other of the devices is configured to receive ultrasound signals from at least one selected from the inside or the outside of the body, in particular from the external device.

65. The system according to claim 59, the device(s) for locating and, where appropriate, tracking, being connected to the electronic control unit and, where appropriate, to a unit for at least one of acquiring or recording the ultrasound signals received and, where appropriate, to at least one of a monitoring or warning unit and, where appropriate, to a battery, the device(s) and the connected unit(s) being housed in a case to be worn by the human or animal.

66. The system according to claim 65, the autonomous case being configured to be connected by wired connection or by a wireless communication protocol such as wifi, NFC, Bluetooth, to an external computing or imaging system.

67. A medical kit comprising:—a system according to claim 59; and
an implant to be implanted in or on a human or animal body.

68. A method of treatment with a device according to claim 48, the method comprising one or more of the steps selected from the group of:
monitoring the spatial evolution of an implant;
monitoring movement or degradation kinetics of an implant when it is evacuated via the natural tract;
tracking fiduciary markers;
determining the positioning of invasive devices during surgical operations;
extracting physiological data from a human or animal body;
determining the positioning of a device for delivering one or more drugs or markers, tracers or materials into the human or animal body;
at least one of activating or controlling a device internal to an implant; and
activating a device for stimulating a peripheral nerve.
